(19) United States
(12) Patent Application Publication
(10) Pub. No.: US 2026/0335390 A1
(43) Pub. Date: Oct. 29, 2026

(54) PEST CONTROL AGENT-INFUSED FISH FEED

(71) Applicant: CAN TECHNOLOGIES, INC., Wayzata, MN (US)

(72) Inventors: David A. COOK, Coon Rapids, MN (US); Thomas GITLESEN, Kleppe (NO)

(73) Assignee: CAN TECHNOLOGIES, INC., Wayzata, MN (US)

(21) Appl. No.: 19/124,410

(22) PCT Filed: Oct. 26, 2023
(86) PCT No.: PCT/US2023/077889

§ 371 Date: Apr. 25, 2025

Related U.S. Application Data
(60) Provisional application No. 63/381,362, filed on Oct. 28, 2022.

(51) Int. Cl. A61K 36/58; A23K 20/158; A23K 50/80; A61K 9/00; A61K 31/352; A61P 33/14

(52) U.S. Cl. CPC A61K 36/58; A23K 20/158; A23K 50/80; A61K 9/0056; A61K 31/352; A61P 33/14

(57) ABSTRACT

The present disclosure provides a fish feed and methods for making a fish feed. The fish feed includes a base feed having a porous matrix distributed throughout and a pest control agent composition, where the pest control agent composition includes a pest control agent and one or more oils. The pest control agent composition of the fish feed can be distributed throughout the porous matrix. The pest control agent can include a neem extract rich in azadirachtin A that is distributed throughout the porous matrix of the base feed by a vacuum in fusion process. Other aspects are also included herein.

### CROSS-REFERENCE TO RELATED APPLICATIONS

This application claims the benefit of U.S. Provisional Application No. 63/381,362, filed Oct. 28, 2023, and entitled “PEST CONTROL AGENT-INFUSED FISH FEED,” which is incorporated by reference herein in its entirety.

### FIELD OF THE INVENTION

The present disclosure is related to a fish feed having one or more pest control agents distributed throughout a porous matrix thereof, and compositions and methods for making such fish feeds.

### BACKGROUND

Aquaculture of various fish species provides a critical food source for a growing world population. In 2021, it was estimated that about 175 million metric tons of fish were produced for global consumption. An increase in demand for fish by consumers has resulted in an increase in the number of aquaculture farms around the world. With an increase in aquaculture farms comes an increased need for fish feeds to nourish and maintain the volume fish being produced and for sustainable technologies for safe and effective pest control in farmed fish populations.

While various physical delousing systems and chemotherapeutic agents exist for the management of various pests, many undesirable side effects from these systems and agents can result in significant losses for fish farms. Some estimates indicate that the global aquaculture industry loses greater than $750 million USD a year due to the need to purchase agents such as parasiticides and expensive equipment, lost productivity from starvation and underfeeding associated with physical delousing systems, excess mortality due to physical delousing systems, and the further need to invest in personnel time for the management, control, and research of new parasite management and control methods. In many cases, farmers must monitor their fish to keep sea lice within pre-determined thresholds set by each region, and when necessary, they must cull fish to prevent suffering and the spread of the parasites, further contributing to economic losses.

One avenue for controlling and preventing pest infections and infestations is to provide one or more pest control agents in a fish feed to be consumed by the fish, where the pests eventually consume the pest control agent as they feed on the fish. The manufacture of fish feeds by various techniques can require high temperatures and high shear force applied to the raw materials prior during processing. While the raw materials are able to withstand high processing temperatures and shear forces, various functional agents and active agents, such as various pest control agents, can be susceptible to degradation at such temperatures and forces. Further, many functional agents or active agents are not soluble in various food-grade solutions and thus require more sophisticated mechanisms to incorporate them into a fish feed. Thus, a need exists to provide fish feeds and methods for incorporating functional agents or active agents into the fish feeds using processing that does not lead to degradation of the functional agents or active agents.

### SUMMARY

The present disclosure provides a fish feed including a base feed having a porous matrix distributed throughout, and a pest control agent composition. The pest control agent composition includes a pest control agent and one or more oils, where the pest control agent composition is distributed throughout the porous matrix of the base feed.

In an aspect, the pest control agent includes neem extract rich in azadirachtin A.

In an aspect, the neem extract rich in azadirachtin A includes at least from 15 wt. % to 33 wt. % azadirachtin A.

In an aspect, the one or more oils includes one or more of soy oil, grape seed oil, canola oil, sunflower seed oil, safflower oil, rapeseed oil, hydrogenated rapeseed oil, linseed oil, camelina oil, flaxseed oil, coconut oil, fish oil, poultry oil, or fish oil stearin, or fractions thereof.

In an aspect, the porous matrix is formed by extrusion processing of raw materials including a protein and a starch.

In an aspect, the pest control agent composition is applied to the base feed via a vacuum infusion process.

In an aspect, the pest control agent is at least partially distributed throughout the porous matrix of the base feed.

In an aspect, the pest control agent composition is further coated on an exterior surface of the base feed.

In an aspect, the fish feed includes between about 15 wt. % and about 65 wt. % protein and between about 10 wt. % and about 45 wt. % fat, and where the fish feed pellet includes land-animal protein, fish meal, plant-based protein, or combinations thereof.

In an aspect, the base feed includes one or more of pellets, extruded nuggets, steam pellets, flakes, tablets, or powders.

In an aspect, the pest control agent includes a solid form suspended in one or more oils.

The present disclosure provides a method for making a fish feed. The method can include providing a protein and starch mixture and subjecting it to and extrusion process to produce a base feed having a porous matrix distributed throughout. The method can include creating a pest control agent composition by mixing together a pest control agent and one or more oils to create a suspension. The method can include applying the pest control agent composition to the base feed using a vacuum infusion process to distribute the pest control agent composition throughout the porous matrix of the base feed.

In an aspect, the pest control agent of the methods herein includes a neem extract rich in azadirachtin A including at least from 15 wt. % to 33 wt. % azadirachtin A.

In an aspect, the one or more oils used in the methods herein include one or more of soy oil, grape seed oil, canola oil, sunflower seed oil, safflower oil, rapeseed oil, hydrogenated rapeseed oil, linseed oil, camelina oil, flaxseed oil, coconut oil, fish oil, poultry oil, or fish oil stearin, or fractions thereof.

In an aspect, the extrusion process includes extrusion cooking of raw materials including a protein and a starch.

In an aspect, the method of producing a base feed by an extrusion process followed by applying a pest control agent composition by a vacuum infusion process produces a fish feed having a coefficient of variation of less than 10%.

The present disclosure provides a fish feed product according to the processes herein.

The present disclosure provides a fish feed for reducing, preventing, or controlling a parasite infection or infestation in fish, where the fish feed includes a base feed including a porous matrix distributed throughout; and a pest control agent composition, the pest control agent composition including a pest control agent and one or more oils; and where the pest control agent composition is distributed throughout the porous matrix of the base feed

### BRIEF DESCRIPTION OF THE FIGURES

The drawings illustrate generally, by way of example, but not by way of limitation, various aspects discussed herein. Aspects may be more completely understood in reference to the following drawings, in which:

FIG. 1 is a schematic diagram of a fish feed in accordance with various aspects herein.

FIG. 2 is a schematic diagram of a fish feed in accordance with various aspects herein.

FIG. 3 is a schematic diagram of the lifecycle of the salmon louse Lepeophtheirus salmonis in accordance with various aspects herein.

FIG. 4 is a flow diagram of a method in accordance with various aspects herein.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

As discussed above, there exists a need for incorporating various functional agents or active agents throughout a fish feed. While in some instances such agents can be coated onto the exterior of fish feeds, such exterior applications can result in loss of the agents from the surface during storage, transportation, and direct and prolonged exposure to moisture or humidity. Other instances include adding a functional agent or active agent directly to raw materials prior to forming a fish feed. However, many functional agents and active agents are heat sensitive and susceptible to degradation at high processing temperatures and shear forces, and further have limited solubility for coating applications. Thus, it is not optimal to incorporate various functional agents or active agents into a fish feed during such processing steps due to the limitations imparted by the physiochemical properties of such agents. The present disclosure relates to fish feeds having distributed throughout one or more functional agents or active agents, such as one or more pest control agents. The present disclosure further discloses various methods for producing fish feeds having distributed throughout one or more functional agents or active agents, such as one or more pest control agents, where the methods for producing such fish feeds do not lead to significant degradation of the agents during processing.

As used herein, the term “infection” can refer to a condition where a pestilent organism, including the various pests defined elsewhere herein, can invade any internal or external portion of a host organism's body such that the host organism experiences harm, and where the pestilent organism uses components of the host organism to sustain itself, reproduce, or colonize the host organism.

As used herein, the term “infestation” can refer to the presence of an abnormally large number of pests as defined herein, where the pests are concentrated in a region in numbers that can cause damage or disease through infection of a host organism.

As used herein, the term “pest” can refer to any organism that is detrimental to the health, value, or appearance of another organism. The term pest can include, but is not to be limited to, one or more of various parasites including worms, helminths, flukes, lice, mites; one or more species of bacteria; one or more viruses; one or more type of fungi; and various protozoa (e.g., amoeba).

As used herein, the term “parasite” can refer to one or more species of ectoparasite or endoparasite. As used herein, the term “endoparasite” can refer to organisms that inhabit one or more internal niches of another organism. For example, an endoparasite can inhabit one or more of the tissues, organs, or systems of a host organism. For example an endoparasite can inhabit the gut, blood, or both, of a host organism. As used herein, the term “ectoparasite” can refer to organisms that inhabit or occupy an external niche of another species. For example, an ectoparasite can inhabit or occupy the surface of a host species. In the case of fish, ectoparasites can inhabit the skin of the fish where they sometimes lodge between scales, and they further can feed off of the mucus, blood, skin, gills, muscle, or any combination thereof. Ectoparasites can include species belonging to the phylum Arthropoda. As such, the term ectoparasites further can include crustaceans and one or more species of sea lice that inhabit fish hosts. In an aspect, the ectoparasites can include one or more species of copepod.

As used herein, the term “pest control agent” can refer to an agent for reducing, preventing, or controlling an infection or infestation caused or contributed to by one or more pests. In various aspects, the pest control agents described herein can refer to an agent for reducing, preventing, or controlling an infection or infestation caused by one or more endoparasites or ectoparasites.

### Fish Feeds

The present disclosure provides fish feeds to be used as fish diets in aquaculture applications. The fish feeds herein can include a base feed. The base feed can include a porous matrix distributed throughout the base feed. In various aspects, the fish feed can further include a pest control agent composition, where the pest control agent composition includes one or more pest control agents and one or more oils. A base feed can be formed from various raw materials as described elsewhere herein.

In various aspects, the pest control agent composition can be distributed throughout the porous matrix of the base feed. Pest control agent compositions are further described elsewhere herein. In some aspects, the base feed herein can include a pest control agent composition that is at least partially distributed throughout the porous matrix of the base feed, or completely distributed throughout the porous matrix of the base feed. In various aspects, the pest control agent composition is further coated on an exterior surface of the base feed. The base feed can be at least partially coated on an exterior surface with a pest control agent composition or completely coated on an exterior surface with a pest control agent composition. In various aspects, the fish feeds herein can be at least partially coated with a pest control agent composition on an exterior surface and further can have a pest control agent composition at least partially dispersed throughout the porous matrix of the base feed. In various aspects, the pest control agent composition is coated on an exterior surface of the base feed. The pest control agent is distributed throughout the porous matrix of the base feed either partially or completely by a vacuum infusion process. Similarly, the pest control agents herein are coated on an exterior surface of the base feed either partially or completely by a vacuum infusion process. Alternatively, the coating on an exterior surface of the base feed can be distributed by a spray coating, dip coating, rolling, or electrospray coating process.

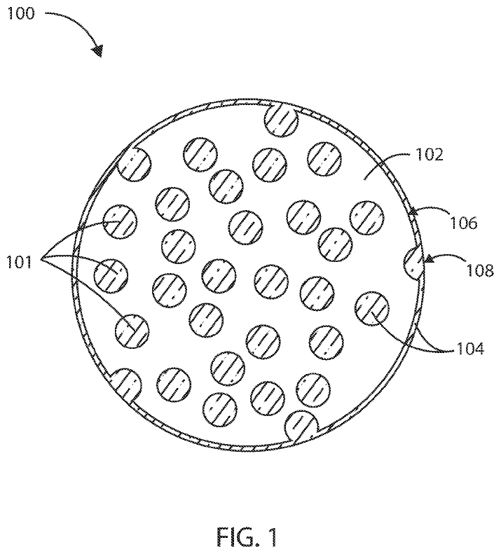

Referring now to FIG. 1, a schematic diagram of a fish feed is shown in accordance with various aspects herein. The fish feed 100 can include a porous matrix distributed throughout the fish feed 100, where the porous matrix includes a plurality of pores 101. It will be appreciated that the processing of the fish feed will result in a distribution of pore sizes such that not all of the pores will be uniform in diameter or distribution throughout the fish feed 100. The fish feed 100 can include a base feed 102 and a pest control agent composition 104 distributed throughout the base feed. The pest control agent composition further can be coated on an exterior surface 106 of the base feed 102. It will be appreciated that the pest control agent composition can include one or more pest control agents and one or more oils, as described elsewhere herein. It will further be appreciated that the fish feed 100 can include a pest control agent composition 104 distributed throughout the base feed 102 and coated on an exterior surface 106 of the base feed 102, and further can include a second pest control agent composition (not shown) coated on an exterior surface 108 of the pest control agent composition 104. It will be appreciated that multiple pest control agent composition coatings can be added to the base feed after the initial pest control agent composition 104 has been added. It will further be appreciated that when multiple pest control agent compositions are added to the base feed, each pest control agent composition can contain a different pest control agent and/or different oil composition as described elsewhere herein.

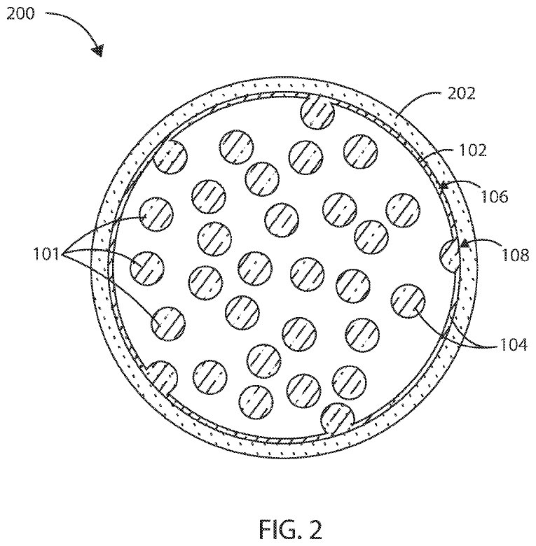

In various aspects, the fish feed 100 can further include one or more coating compositions lacking a pest control agent and distributed on an exterior surface 108 of the pest control agent composition 104. Referring now to FIG. 2, a schematic diagram of a fish feed 200 is shown in accordance with various aspects herein. Fish feed 200 contains like numbered features of fish feed 100, as shown in FIG. 1, and additionally includes a coating composition 202 lacking a pest control agent. The coating composition 202 can include an oil-based composition. The coating composition can include one or more oils and one or more solid particles. The oil in the coating composition can include, but is not to be limited to vegetable oil, canola oil, poultry oil, rapeseed oil, fish oil, soy oil, linseed oil, camelina oil, fish oil stearin, coconut oil, hydrogenated rapeseed oil, and any combinations or fractions thereof. The solid particles can include, but are not to be limited to, wheat gluten, soy protein concentrate, pea protein concentrate, fish meal, concentrated fish soluble protein, feather meal, bentonite, diatomaceous earth, zeolite, ammonium phosphate, dicalcium phosphate, monocalcium phosphate, algae oil, and any combinations thereof.

In various aspects, the coating composition is coated on an exterior surface of the pest control agent composition. The fish feed can be at least partially coated with the coating composition on an exterior surface of the pest control agent composition or completely coated with the coating composition on an exterior surface of the pest control agent composition. In various aspects, the coating composition can form at least two layers surrounding the fish feed. The coating composition may be in the form of a slurry. In various aspects, the coating composition slurry can be made by adding the one or more solid particles to a volume of oil to form the coating composition slurry. The coating composition slurry can be used to coat the fish feed or fish feed product in at least two, at least three, at least four, or more layers. In various aspects, the coating composition can serve the purpose to prevent leakage from the fish feed, including preventing leakage of the pest control agent composition from the fish feed.

It will be appreciated that the fish feeds herein can include suitable types of fish feed specific for a given fish species. The fish feeds can be used as a component of a diet fed to any species belonging to the families Cyprinidae, Cichlidae, Pangasiidae, Sciaenidae, Serranidae, Carangidae, Sparidae, Lateolabracidae, Moronidae, Mugilidae, Cypriniformes, Latidae, Eleotridae, Tilapiini and Salmonidae. In various aspects, this disclosure provides a fish feed or fish feed diet for species within the family Salmonidae. The fish feeds provided herein can be used to feed wild fish or farmed fish. In various aspects, both wild fish and farmed fish can be fed simultaneously. Further, the fish feed can be used to feed freshwater fish or salt water (e.g., marine) fish, or both.

The fish feeds of the present disclosure can be produced by creating a base feed using raw materials that can be chosen based on the application in which it is to be used and on the fish species. In various aspects, the fish feed is a solid fish feed. In other aspects, the fish feed can include both a solid fish feed component and a liquid fish feed component. Base feeds can include pellets, extruded nuggets, steam pellets, flakes, tablets, powders, and the like. In various aspects, the base feed can include a base feed pellet. In various aspects, the fish feeds can include an extruded, pressed, or particulate base feed. In some aspects, the base feed can include a porous matrix distributed throughout. Fish feeds including a liquid fish feed component can include aqueous solutions, oils, oil and water emulsions, slurries, suspensions, and the like. In various aspects, a solid fish feed can further include one or more oils disposed on the surface or distributed throughout a porous matrix present in the fish feed. In various aspects, one or more pest control agents can be suspended in one or more oils and disposed on the surface or distributed throughout a porous matrix present in the fish feed.

The fish feeds herein can include a number of different ingredients or raw materials that can sustain life, growth, and reproduction of the fish. The fish feeds can include any substrate that is edible to fish. For example, an edible substrate can provide a source of nutrition to the fish or can be an inert substrate with no nutritive value to the fish. In various aspects, the fish feeds herein can include feeds that are either nutritional fish feeds or non-nutritional feeds. Nutritional fish feeds can include a nutritional food stuff formulated for fish as part of its diet as the main source of nutrition, growth, and reproduction. Suitable nutritional fish feeds can include one or more of proteinaceous material as a source of proteins, peptides, and amino acids; carbohydrates; and fats, as described below. Non-nutritional fish feeds can include any substrate that is edible to fish but does not provide nutrition to sustain life, growth, or reproduction. In various aspects, the nutritional or non-nutritional fish feeds herein can include one or more compounds designed to alter the quality, quantity, or appearance of a fish and fish tissue. For example, a nutritional or non-nutritional fish feed can include a carotenoid compound to improve the appearance (e.g., color) of the muscle tissue. By way of example, the carotenoid compound can include compounds such as astaxanthin.

The fish feeds herein can include a complete fish feed. A complete fish feed can include a feed for fish that is compounded to be fed as the sole ration and that can maintain life, promote growth, and sustain reproduction without any additional substances being consumed except water. Complete fish feeds can include compounded mixtures containing various energy sources such as carbohydrates, proteins, and fats. In various aspects, the fish feeds herein can include at least a protein and a starch. Additional ingredients can be included, such as vitamins and minerals as necessary to support the life, growth, and reproduction of fish. A complete fish feed can include ingredients such as, but not limited to, fish meal, poultry meal, plant meal, vegetable meal, corn meal, corn gluten meal, soy meal, soy protein concentrate, single cell protein, insect meal, algae meal, algae oil, krill meal, krill oil, meat meal, blood meal, feather meal, starches, tapioca starch, wheat, wheat gluten, guar meal, guar protein concentrate, peas, pea protein concentrate, pea starch, beans, faba beans, sunflower meal, vegetable oil, canola oil, poultry oil, rapeseed oil, fish oil, soy oil, linseed oil, camelina oil, lecithin, macro-minerals, minerals, vitamins, amino acids, pigments, and any combinations thereof. It will be appreciated that the fish feeds herein can include fish meal that can include plant or animal derived matter. Any animal derived matter present in the fish meal can be derived from the same species of fish or a different species of fish (e.g., heterologous species or non-fish species).

The total protein content in the fish feed can be from 10 wt. % to 70 wt. %, from 15 wt. % to 65 wt. %, from 20 wt. % to 60 wt. %, or from 25 wt. % to about 55 wt. %. The total protein in the fish feed can be at least 10%, 15%, 20%, 25%, 30%, 35%, 38%, 40%, 42%, 44%, 46%, 48%, 50%, 52%, 55%, 60%, 65%, or at least 70% by weight, or any amount within a range of any of the forgoing. The total protein in the fish feed can be variable depending on the formulation, species, age, and intended use of the feed. It will be appreciated that the various protein requirements of the species of fish receiving the fish feed can be adjusted to meet the protein requirements of that species.

The protein in the fish feeds herein can be from any suitable source including, but not limited to, one or more of fish meal, land-animal protein (e.g., poultry meal), plant-based protein (e.g., vegetable meal), or any combinations thereof. The fish feed can include from 0% to 80%, from 10% to 80%, from 20% to 75%, from 30% to 70%, from 60% to 80%, or from 10% to 30%, or at least 10%, 20%, 30%, 40%, 50%, 60%, 70%, or 75% fish meal by weight, or any amount within a range of any of the forgoing. The fish feed can include from 0% to 80%, from 10% to 80%, from 20% to 75%, from 30% to 70%, from 60% to 80%, or from 10% to 30%, or at least 10%, 20%, 30%, 40%, 50%, 60%, 70%, or 75% land-animal protein by weight, or any amount within a range of any of the forgoing. The fish feed can include between 0% to 80%, from 10% to 80%, from 20% to 75%, from 30% to 70%, from 60% to 80%, or from 10% to 30%, or at least 10%, 20%, 30%, 40%, 50%, 60%, 70%, or 75% plant-based protein by weight, or any amount within a range of any of the forgoing.

Total fat (e.g., oil, fat, and/or lipids) in the fish feed can be from 5% to 50%, from 10% to 45%, from 15% to 40%, or from 20% to 35%. The total fat in the fish feed can be at least 5%, 10%, 15%, 20%, 25%, 30%, 35%, 40%, 45%, or at least 50% by weight, or any amount within a range of any of the forgoing. The total fat in the fish feed can be variable depending on the formulation, target fish species, and intended use of the fish feed. It will be appreciated that the various fat requirements of the species of fish receiving the fish feed and can be adjusted to meet the fat requirements of that species. Suitable fats for use herein can include, but are not to be limited to, those provided by canola oil, poultry oil, rapeseed oil, fish oil, soy oil, linseed oil, camelina oil, palm oil, lecithin, or any combinations or fractions thereof.

The moisture content of the fish feeds herein can vary depending on the contents and preparation method of the feed. In various aspects, the moisture content can be from 1% to 20%, from 2% to 18%, from 5% to 15%, or from 6% to 12% by weight.

In addition to pest control agents disposed throughout the porous matrix of the fish feed or coated on an exterior surface of the fish feed, the fish feeds herein can include one or more pest control agents added to the base feed, provided that the pest control agents can withstand various processing temperatures and shear forces. In various aspects, the one or more pest control agents can be present in the base feed in an amount effective to produce an inhibitory effect on one or more pests, as will be described elsewhere herein. As such, the base feeds herein can further include pest control agents for controlling infections or infestations caused by one or more pests. In various aspects, the base feeds herein can include pest control agents for controlling endoparasitic or ectoparasitic infections or infestations. In various aspects, the parasitic infection or infestation is a copepod infection or infestation. In some aspect, the parasitic infection or infestation is a sea lice infection or infestation. Various pests suitable as targets for the pest control agents herein are described elsewhere.

Each pest control agent included within the base feeds or fish feeds herein can be individually capable of controlling one or more of a parasitic, bacterial, viral, fungal, or protozoal infections or infestations. Therefore, it should be understood that any given pest control agent for use in the many aspects described herein can be referred to as exhibiting one or more inhibitory effects, including antiparasitic effects (e.g., anti-ectoparasitic, anti-endoparasitic), antibacterial effects, antiviral effects, antifungal effects, or antiprotozoal effects. In various aspects, the inhibitory effect can include an antiparasitic effect, where the antiparasitic effect can further include an anti-ectoparasitic effect, an anti-endoparasitic effect, or both. It will be appreciated that the inhibitory effects can result in reducing, preventing, or controlling the concentration and spread of the various parasitic, bacterial, viral, fungal, or protozoal organisms described herein. In various aspects, a pest control agent of the present disclosure can produce inhibitory effects against one or more pests including one or more effects for reducing, preventing, or controlling the concentration and spread of various endoparasites or ectoparasites. Reducing, preventing, or controlling the parasites can include complete prevention of infection or infestation in the fish population or on each fish, a reduction in the total number of parasites present in the fish population or on each fish, or controlling how many parasites are present in the fish population or on each fish according to local regulatory requirements. It will be appreciated that the inhibitory effects described herein can be measured against a population fish infected or infested with one or more pests that are fed a diet lacking the neem extract rich in azadirachtin A.

In other aspects, the inhibitory effect against the pests can include one or more of an anti-feedancy effect, an anti-molting effect, an antifertility and anti-fecundity effect, or an antiparasitic effect. As used herein, “anti-feedancy effect” can refer to an effect exerted by one or more pest control agents that stops or inhibits feeding by the pests resulting in their malnourishment, delayed development, prevention or delay of molting, and death. Malnourished sea lice are also less likely to efficiently immunomodulate their hosts, and thus they are less capable evading host immunity. As used herein, the term “anti-molting” can refer to an effect exerted by one or more pest control agents that prevents or delays the process of molting in the pests. The process of molting occurs as pests grow and shed their exoskeletons from one life stage to the next and is controlled hormonally and neuronally, and the pest control agents herein can exert one or more anti-molting effects against the pests. As used herein, the terms “antifertility effect” and “anti-fecundity effect” are referred to collectively as an “antifertility and anti-fecundity effect” and can include one or more effects on male or female reproduction. It will be appreciated that the term “fertility” can refer to the actual number of offspring born to or eggs released from a female, and the term “fecundity” can refer to the biological potential for reproduction, and due to their close relationship the two terms as used herein can be used interchangeably unless otherwise noted. An antifertility and anti-fecundity effect can include a reduction in total gamete production in males and females, a complete or partial inhibition of viable egg production, a change in the anatomy and morphology of the gametes of males or females, a change in the potential for egg fertilization, and a reduction in the total number of gravid female pests.

In an administered form, the fish feeds herein can include an amount of pest control agent at from about 0.01-100 grams of pest control agent per kilogram fish feed (g/kg), about 90 g/kg fish feed, about 80 g/kg fish feed, about 70 g/kg fish feed, about 60 g/kg fish feed, about 50 g/kg fish feed, about 40 g/kg fish feed, about 30 g/kg fish feed, about 20 g/kg fish feed, about 1-10 g/kg fish feed, about 2-9 g/kg fish feed, about 3-7 g/kg fish feed, about 4-6 g/kg fish feed, or about 5 g/kg fish feed. The total amount of pest control agent can include a pest control agent found in the base feed, pest control agent found in one or more pest control agent compositions infused into the porous matrix of the base feed, pest control agent compositions coated on an exterior surface of the base feed, or any combinations thereof.

In various aspects, the fish feeds herein can include an amount of pest control agent and/or active ingredient in an amount effective to produce an inhibitory effect against one or more pests, including a concentration from about 0.01 g/kg fish feed, 0.05 g/kg fish feed, 0.1 g/kg fish feed, 0.2 g/kg fish feed, 0.3 g/kg fish feed, 0.4 g/kg fish feed, 0.5 g/kg fish feed, 0.6 g/kg fish feed, 0.7 g/kg fish feed, 0.8 g/kg fish feed, 0.9 g/kg fish feed, 1.0 g/kg fish feed. 1.25 g/kg fish feed, 1.5 g/kg fish feed, 1.75 g/kg fish feed, 2.0 g/kg fish feed, 2.25 g/kg fish feed, 2.5 g/kg fish feed, 2.75, g/kg fish feed, 3.0 g/kg fish feed, 5.0 g/kg fish feed. 5.25 g/kg fish feed, 5.5 g/kg fish feed, 5.75 g/kg fish feed, 6.0 g/kg fish feed, 6.25 g/kg fish feed, 6.5 g/kg fish feed, 6.75, g/kg fish feed, 7.0 g/kg fish feed, 7.0 g/kg fish feed. 7.25 g/kg fish feed, 7.5 g/kg fish feed, 7.75 g/kg fish feed, 8.0 g/kg fish feed, 8.25 g/kg fish feed, 8.5 g/kg fish feed, 8.75, g/kg fish feed, 9.0 g/kg fish feed, 9.25 g/kg fish feed, 9.5 g/kg fish feed, 9.75, g/kg fish feed, 10.0 g/kg fish feed, 15 g/kg fish feed, 20 g/kg fish feed, 25 g/kg fish feed, 30 g/kg fish feed, 35 g/kg fish feed, 40 g/kg fish feed, 45 g/kg fish feed, 50 g/kg fish feed, 55 g/kg fish feed, 60 g/kg fish feed, 65 g/kg fish feed, 70 g/kg fish feed, 75 g/kg fish feed, 80 g/kg fish feed, 85 g/kg fish feed, 90 g/kg fish feed, 95 g/kg fish feed, or 100 g/kg fish feed, or any amount within a range of any of the forgoing concentrations. The total amount of pest control agent can include a pest control agent found in the base feed, pest control agent found in one or more pest control agent compositions infused into the porous matrix of the base feed, pest control agent compositions coated on an exterior surface of the base feed, or any combinations thereof.

It will be appreciated that the aforementioned concentrations equate to an amount from about 0.001-10 weight percent (% w/w) total pest control agent to fish feed. In various aspects, the fish feeds herein can include an amount of pest control agent effective to produce an inhibitory effect against one or more pests including from 0.001% w/w, 0.0020% w/w, 0.003% w/w, 0.004% w/w, 0.005% w/w, 0.006% w/w, 0.007% w/w, 0.008% w/w, 0.009% w/w, 0.010% w/w, 0.020% w/w, 0.030% w/w, 0.040% w/w, 0.050% w/w, 0.060% w/w, 0.070% w/w, 0.080% w/w, 0.090% w/w, 0.10% w/w, 0.11% w/w, 0.12% w/w, 0.13% w/w, 0.14% w/w, 0.15% w/w, 0.16% w/w, 0.17% w/w, 0.18% w/w, 0.19% w/w, 0.20% w/w, 0.21% w/w, 0.22% w/w, 0.23% w/w, 0.24% w/w, 0.25% w/w, 0.26% w/w, 0.27% w/w, 0.28% w/w, 0.29% w/w, 0.30% w/w, 0.31% w/w, 0.32% w/w, 0.33% w/w, 0.34% w/w, 0.35% w/w, 0.36% w/w, 0.37% w/w, 0.38% w/w, 0.39% w/w, 0.40% w/w, 0.41% w/w, 0.42% w/w, 0.43% w/w, 0.44% w/w, 0.45% w/w, 0.46% w/w, 0.47% w/w, 0.48% w/w, 0.49% w/w, 0.50% w/w, 0.51% w/w, 0.52% w/w, 0.53% w/w, 0.54% w/w, 0.55% w/w, 0.56% w/w, 0.57% w/w, 0.58% w/w, 0.59% w/w, 0.60% w/w, 0.61% w/w, 0.62% w/w, 0.63% w/w, 0.64% w/w, 0.65% w/w, 0.66% w/w, 0.67% w/w, 0.68% w/w, 0.69% w/w, 0.70% w/w, 0.71% w/w, 0.72% w/w, 0.73% w/w, 0.74% w/w, 0.75% w/w, 0.76% w/w, 0.77% w/w, 0.78% w/w, 0.79% w/w, 0.80% w/w, 0.81% w/w, 0.82% w/w, 0.83% w/w, 0.84% w/w, 0.85% w/w, 0.86% w/w, 0.87% w/w, 0.88% w/w, 0.89% w/w, 0.90% w/w, 0.91% w/w, 0.92% w/w, 0.93% w/w, 0.94% w/w, 0.95% w/w, 0.96% w/w, 0.97% w/w, 0.98% w/w, 0.99% w/w, 1.0% w/w, 2.0% w/w, 3.0% w/w, 4.0% w/w, 5.0% w/w, 6.0% w/w, 7.0% w/w, 8.0% w/w, 9.0% w/w, or 10.0% w/w, or any amount within a range of any of the forgoing values. The total amount of pest control agent can include a pest control agent found in the base feed, pest control agent found in one or more pest control agent compositions infused into the porous matrix of the base feed, pest control agent compositions coated on an exterior surface of the base feed, or any combinations thereof.

In various aspects herein, the fish feeds can be administered to the fish having a concentration of pest control agent selected from the group including 0.05% w/w, 0.06% w/w, 0.07% w/w, 0.08% w/w, 0.09% w/w, 0.10% w/w, 0.11% w/w, 0.12% w/w, 0.13% w/w, 0.14% w/w, 0.15% w/w, 0.16% w/w, 0.17% w/w, 0.18% w/w, 0.19% w/w, 0.20% w/w, 0.30% w/w, 0.40% w/w, 0.50% w/w, 0.60% w/w, 0.70% w/w, 0.80% w/w, 0.90% w/w, or 1.0% w/w, pest control agent to fish feed or a range within any of the forgoing concentrations.

### Pest Control Agents

The pest control agents suitable for use in the fish feeds and pest control agent compositions herein can adversely affect pests that feed off their hosts. It will be appreciated that when a host fish has consumed the pest control agents as a component of their daily diet for a given duration, it can be transferred to the body of the pest when that pest takes a meal from the host. The pest control agents can include any functional agent or active agent that affects, facilitates, or contributes to the eradication or reduction of a pest infection or pest infestation of a fish or population of fish. Additionally, suitable pest control agents can alleviate or improve one or more of the symptoms associated with a pest infection or pest infestation, as a result of reducing, preventing, or controlling an infection or infestation. Pest control agents for use herein can be biologically active to one or more fish pests and for one or more fish species.

It will be appreciated that when the fish consume the pest control agents described herein, the pest control agent is systemically distributed throughout the tissues and fluids of the fish. Pests can be exposed to the pest control agents upon ingestion of the pest control agent through the skin, flesh, blood, mucus, mucous membranes, or other tissues of the host organism. Modulation of the pests, such as modulation of the pest behavior and life cycle occur to reduce, prevent, or control the pest infection or infestation in the fish. In various aspects, the pests can be repelled or killed by the pest control agents herein. Thus, the pest control agents herein can be provided to the pests in a fish feed or a pest control agent composition in an amount sufficient to modulate the behavior of the pests.

Modulation of the pests can have many effects on the pest population, including an ultimate reduction in the number of viable pests available to infect or infest the host fish. Modulation of the pests can include a modulation of the mortality of the pests. It will be appreciated that modulation of the mortality in the pests can include a decrease in the number of viable pests present on the fish or in the fish habitat. Modulation of the pests can further include modulation of pest behavior, including a change in feeding habits, a change in feeding patterns, a change in appetite, a change in mobility patterns, a change in swimming and migration patterns, a change in mating patterns, a change in development, a change in fertility, or any combination thereof, as compared to pests found on control fish not fed a pest control agent. The change in feeding patterns can include a decrease in feeding patterns. The change in appetite can include a decrease in appetite. The change in mobility can include a decrease in mobility. The change in swimming and migration patterns can include a decrease in swimming and migration due to lethargy and lack of energy. The change in mating patterns can include a decrease in mating patterns, which in turn can lead to a decrease in development or production of offspring. The change in development can include an inhibition of development due to an inhibition of the molting process leading to a decrease in development in the pests or a delay in development in the pests. The change in fertility can include an inhibition of or delay in egg production, an inability to produce viable eggs, or a reduction in the total number of gravid female pests.

In various aspects, modulation of the pests can include a change in development of the pests through their life cycle, including modulation of growth or progression through a particular life stage, modulation of growth or progression from one life stage to the next life stage (e.g., modulating molting), modulation of egg production, modulation of fertility, or any combination thereof. Modulation of growth or progression through a particular life stage can include halting the growth of the organism and preventing further physical development including a decrease in size or sexual development. In some aspects, modulation of growth or progression from one life stage to the next life stage can include preventing the pests from transitioning from one life stage to the next by inhibiting the molting process. Modulation of egg production can include decreasing the production of eggs by females, which can further result in a decrease in fertility of the adult females. Modulation of fertility can include decreasing the fertility of both female and male pests.

In various aspects, the administration of pest control agents to fish as described herein further can have a beneficial effect on the fish. The administration of the pest control agents can impart a beneficial effect by improving fish welfare by reducing the parasitic load, or total number of parasites, in a given environment around the fish. The administration of the pest control agents can impart a beneficial effect by a reduction in the overall mortality within a fish population by lessening or reducing the impact of a parasitic infection or infestation on the fish population. The administration of the pest control agents can impart a beneficial effect by minimizing or altogether eliminating the impact on the quality and quantity of fish flesh within the fish population.

The fish feeds described herein can include, or be supplemented with, one or more pest control agents. Where a fish feed includes at least two or more different pest control agents, each pest control agent can be individually active (or biologically active) and capable of modulating one or more of the behavior, development, or fertility of a pest. Alternatively, the pest control agents can be a component of a pest control agent composition that can be fed separately to fish. Each pest control agent can be individually effective against one or more different pests as described herein.

Pest control agents suitable for use in the fish feeds and pest control agent compositions herein can include one or more active agents, including synthetic or natural agents. The one or more synthetic or natural agents can include agents classified as an active pharmaceutical ingredient, a veterinary medicinal product, and the like. In some aspects, the active agent for the pest control agents herein can be obtained from a plant belonging to the genus Azadirachta. The pest control agent can be obtained or extracted from Azadirachta indica—a tree commonly known as the “Neem” tree. Extracts prepared from plants belonging to the genus Azadirachta (e.g., Azadirachta indica) can include potent terpenoid compounds, including one or more azadirachtinoids. The azadirachtinoids include azadirachtin compounds such as azadirachtin A, azadirachtin B, azadirachtin D, azadirachtin E, azadirachtin F, azadirachtin G, azadirachtin H, azadirachtin I, azadirachtin K, and/or other azadirachtin variants. The extracts from plants belonging to the genus Azadirachta can also include many other components in various quantities. In some aspects, the extracts can include additional compounds such as the limonoids salannin, nimbin, deacetylsalinin, and 6-desacetylnimbin. In various aspects, the extracts can further include one or more azadirachtinins.

As used herein, the term “azadirachtin” can refer to the collective term applied to a large group of active compounds and is intended to encompass not only all naturally occurring variants or derivatives of azadirachtin, including but not limited to azadirachtins A, B, D, E, F, G, H, I, K, but also all synthetic variants, fragments, analogues, and derivatives thereof. In this regard, it will be appreciated that any azadirachtin variants, fragments, derivatives, or analogues for use herein should be functional, in that they exhibit at least one inhibitory effect as described.

Azadirachtin can be obtained or extracted from any part of the Azadirachta indica plant including, for example, the leaves, stems, bark, fruit, seeds, or any combinations thereof by one or more extraction processes. Suitable methods of extraction can include techniques that exploit mechanical pressing of neem seeds (i.e., kernels) and the use of non-polar solvents. Various solvent extraction techniques exploiting alcohol or an aqueous extraction process, mechanical pressing, and non-polar extraction methods can be used to produce azadirachtin A-rich pest control agents for use herein and are described in U.S. Pat. Nos. 4,556,562; 5,695,763; and 11,096,404; the contents of which are incorporated herein by reference in their entirety.

For example, azadirachtin can be effectively recovered from the seeds of the Neem tree. An exemplary method to recover azadirachtin from neem seeds can include providing neem seeds, crushing the neem seeds, extracting azadirachtin from the crushed seeds with water, and then extracting azadirachtin from the water by adding a second extraction solvent including a non-aqueous solvent that is not miscible with water and has a higher solubility of azadirachtin than water or a surfactant having a turbidity temperature between 20 °C and 80 °C. The concentrated azadirachtin can be recovered from the second extraction solution and shows high activity as an insecticide and parasiticide. Extraction methods employing polar solvents (e.g. water) lead to extracts that are rich in polar components, such as azadirachtin compounds.

In various aspects, the azadirachtin suitable for use herein includes azadirachtin A, which is by its scientific name of dimethyl [2aR-[2aα,3β,4β(1aR*,2S*,3aS*,6aS*,7S*,7aS*),4aβ,5α, 7aS*,8β(E),10β,10aα,10bβ]]-10-(acetyloxy)octahydro-3,5-dihydroxy-4-methyl-8-[(2-methyl-1-oxo-2-butenyl)oxy]-4-(3a,6a,7,7a)-tetrahydro-6a-hydroxy-7a-methyl-2,7-methanofuro[2,3-b]oxireno[e]oxepin-1a(2H)-yl)-1H,7H-naphtho-[1,8-bc:4,4a-c′]difuran-5,10a(8H)-dicarboxylate.

Azadirachtin A is the most abundant of a group of the azadirachtinoids. Azadirachtin A makes up about 80% of the azadirachtinoids in the neem seed kernel. The structural formula of azadirachtin A is:

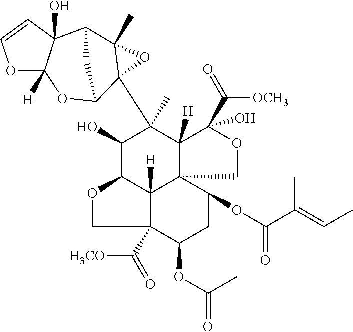

The pest control agents herein can include neem extracts that are an aqueous extract. In various aspects, the neem extract can include an aqueous extract of neem seed. The aqueous extract of neem seed can include an aqueous extract of the neem seed kernel. The aqueous extract of neem seed can include an aqueous extract of the entire neem seed, including the neem seed kernel and the neem seed coating. The aqueous extract of neem seed can be in liquid form, or it can be dried to remove water to create a powder form. By way of example, the neem extracts herein can include an aqueous extract of neem seed or an aqueous extract of neem seed kernel that has been dried into a powder.

It will be appreciated that the pest control agents described herein are not the same thing as neem extracts described as neem oil or solvent-first neem extracts. In various aspects, the pest control agents herein including azadirachtin are richer in the azadirachtinoid active ingredients, and in particular azadirachtin A, than are neem oil and other oil-based formulations. This is due to the fact that azadirachtinoids, such as azadirachtin A, are relatively polar complex terpenoids with a large number of oxygen functionalities, which make the molecules moderately water-soluble (e.g., a solubility of approximately 2 g/L). As a result, azadirachtinoids such as azadirachtin A are present in much higher concentrations in the extracts obtained employing polar solvents than in neem oil or solvent-first neem extracts. Without wishing to be bound by any particular theory, it is believed that the bioavailability of the active ingredients to the target parasite in the water-based extract of azadirachtin A rich extracts of the present disclosure can be greater than in neem oil given the increased solubility and miscibility of the water-based extract in water. Thus, the pest control agents herein do not, comprise, consist, or consist essentially of, neem oil. The pest control agent of the fish feed provided herein can comprise, consist, or consist essentially of azadirachtin A.

The pest control agents including neem extract rich in azadirachtin A can include those having from at least 15 wt. % to 33 wt. % azadirachtin A. In various aspects, pest control agents including neem extract rich in azadirachtin A can include those having from at least 20 wt. % to 26 wt. % azadirachtin A. In various aspects, pest control agents including neem extract rich in azadirachtin A can include those having from at least 28 wt. % to 31 wt. % azadirachtin A. In some aspects, pest control agents including neem extract rich in azadirachtin A can include those having from at least 29 wt. % to 30 wt. % azadirachtin A. In other aspects, pest control agents including neem extract rich in azadirachtin A can include those having from at least 34 wt. % to 40 wt. % azadirachtin A. In various aspects, pest control agents rich in azadirachtin A can include those having from 30+1 wt. % azadirachtin A. In various aspects, pest control agents rich in azadirachtin A can include those having from 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, or 33 wt. %, or any amount falling within a range of any of the forgoing. In yet other aspects, pest control agents including neem extract rich in azadirachtin A can include those having from at least 34 wt. % to 45 wt. % azadirachtin A, or at least 38 wt. % to 43 wt. %. As used herein, the terms “neem extract rich in azadirachtin A” and “azadirachtin A-rich composition” can be used interchangeably unless otherwise noted. A composition of an exemplary neem extract rich in azadirachtin A pest control agent suitable for use herein can include the formula as outlined in Table 1.

TABLE 1
Exemplary Azadirachtin-A Rich Pest Control Agent Formulation

| Azadirachtin Composition | % w/w |
|---|---|
| Azadirachtin A | 15-33 |
| Azadirachtin B | ≤19.0 |
| Azadirachtin D | ≤13.0 |
| Azadirachtin E | ≤5.0 |
| Azadirachtin F | ≤5.0 |
| Azadirachtin G | ≤5.0 |
| Azadirachtin H | ≤5.0 |
| Azadirachtin I | ≤5.0 |
| Azadirachtin K and other Azadirachtins | ≤5.0 |
| Total Azadirachtins | 52.0 |

The pest control agent including a neem extract rich in azadirachtin A can further include other azadirachtinoids at various concentrations. The azadirachtinoids can include azadirachtin compounds such as azadirachtin B at from ≤19.0% w/w, or from ≤6.0% w/w, or from 4.0 to 6.0% w/w, or from 5.6% w/w to 6.0% w/w; azadirachtin D at from ≤6.0% w/w, or from ≤13.0% w/w, or from 2.5 to 5.0, or from 4.0% w/w to 5.0% w/w; azadirachtin E at from ≤5.0% w/w, or from 1.0% w/w to 5.0% w/w, or from 1.5% w/w to 2.0% w/w; azadirachtin F at from ≤5.0% w/w, or from 1.0% w/w to 5.0% w/w, or from 1.5% w/w to 2.0% w/w; azadirachtin G at from ≤5.0% w/w, or from 1.0% w/w to 5.0% w/w, or from 1.5% w/w to 2.0% w/w; azadirachtin H at from ≤5.0% w/w, or from 1.0% w/w to 5.0% w/w, or from 2.5% w/w to 4.0% w/w; azadirachtin I at from ≤5.0% w/w, or from 1.0% w/w to 4.0% w/w, or from 1.5% w/w to 2.5% w/w; and azadirachtin K and/or other azadirachtin variants at from ≤5.0% w/w, or from 1.0% w/w to 5.0% w/w, or from 2.5% w/w to 4.0% w/w. The extracts further can include azadirachtinin at from ≤5.0% w/w, or from 1.0% w/w to 5.0% w/w, or from 2.5% w/w to 4.0% w/w.

An exemplary pest control agent suitable for use herein can include an aqueous extract of neem seed that has been dried into a powder. The powder can include the appearance of a fine white powder. The exemplary pest control agent can include azadirachtin A at a concentration of from 17 wt. % to 37 wt. %, azadirachtin B at a concentration of from 0 wt. % to 19 wt. %, and azadirachtin D at a concentration of rom 0 wt. % to 13 wt. %. The exemplary pest control agent further can include trace amounts of other limonoids including nimbin and salannin.

Exemplary pest control agents including azadirachtin A rich compositions include, but are not to be limited to, NeemAzal® (Coromandel, Intl. Ltd., Telangana, India) or NeemAzal® Technical (Coromandel, Intl. Ltd., Telangana, India), or any derivatives of combinations thereof.

### Method of Making a Fish Feed

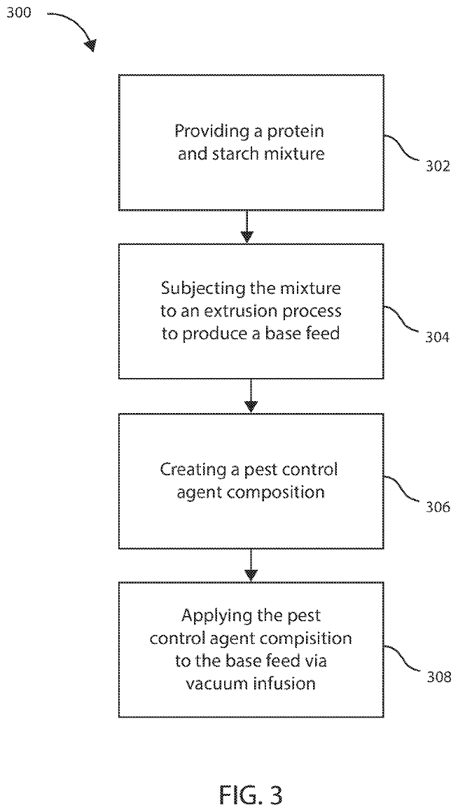

The disclosure herein provides a method for making fish feeds including one or more pest control agents. Referring now to FIG. 3, a method 300 for making a fish feed as described is shown in accordance with various aspects herein. The method 300 can include making a fish feed infused with a quantity of pest control agent composition. The method 300 can include making a fish feed by providing at least a protein and starch mixture at 302. The method 300 can include subjecting the protein and starch mixture to an extrusion process to produce a base feed having a porous matrix distributed throughout at 304. The method can include creating a pest control agent composition at 306 by mixing together a pest control agent and one or more oils to create a suspension. The method can include applying the pest control agent composition to the base feed at 308 using a vacuum infusion process to distribute the pest control agent composition throughout the porous matrix of the base feed. The disclosure herein further can include a fish feed manufactured according to the methods described herein. The method further can include the step of sealing the fish feed to prevent nutrient and pest control agent leakage by applying a coating of fish oil to the fish feed after the vacuum infusion process. Any sealing substance used to seal the pest control agent composition can be applied such that it coats all or a part of a surface of the fish feed. In various aspects, the extrusion process includes an extrusion cooking process. In various aspects, the methods herein are suitable for making a fish feed product according to the steps recited herein.

In various aspects, the base feeds herein can be made using an extrusion process or a pressing process. It will be appreciated that processing a base feed can include cooking the raw materials at temperatures and shear forces that exceed the stability of the pest control agents described herein. In many aspects, it is not desirable to include heat labile pest control agents during the extrusion process or a pressing process due to degradation of the compounds at high processing temperatures and under high shear forces. Thus, the present disclosure provides for the use of a separate vacuum infusion step to incorporate the pest control agent composition into the base feed after processing. The vacuum infusion step can be performed after the initial extrusion or pressing of the base feed. In some aspects, the vacuum infusion step can be performed immediately following the extrusion or pressing of the base feed. In other aspects the vacuum infusion step can be performed at a predetermined time after the extrusion or pressing process. It will be appreciated that the vacuum infusion step allows the oil and pest control agent suspension to be incorporated into the pores of the base feed. The oil and pest control agent suspension can be maintained within the pores via various forces, including steric or geometric restriction, Keppler forces, capillary action, and the like. As a result of using the oil and pest control agent suspension in the infusion process, the amount of evaporation from within the pores of the base feed is minimal, and thus less of the pest control agent is lost during storage, transportation, and handling.

One advantage of performing the vacuum infusion step after the extrusion process or pressing process is that there is limited to no degradation of the functional or active agent when incorporated into the base feed as a separate and distinct processing step. An additional important advantage of performing the vacuum infusion step after the extrusion process or pressing process is that the functional agent or active agent can be added to the base feed using equipment that is separate from the main manufacturing equipment used to make the base feed. This advantage allows for a more streamlined manufacturing process, tailored temperature control, a dedicated or isolated processing line for addition of the pest control agent compositions, a reduction or elimination of cross contact of the manufacturing equipment used to make base feed with any functional or active agents, and more simplified cleaning routines for both the base feed manufacturing equipment and the equipment used for the vacuum infusion process.

The methods herein can generate fish feeds containing one or more pest control agents distributed throughout the porous matrix of the base feed or about an exterior surface of the base feed. It will be appreciated that the fish feeds described herein can include a pest control agent that is at least partially dispersed throughout the porous matrix of the base feed. In various aspects, the fish feeds herein can include a pest control agent that is at least completely dispersed throughout the porous matrix of the base feed. In some aspects, the fish feeds herein can be at least partially coated on an exterior surface of the base feed with pest control agent or completely coated on an exterior surface of the base feed with the pest control agent composition. In various aspects, the fish feeds herein can include a pest control agent that is at least partially coated on an exterior surface of the base feed with pest control agent and at least partially dispersed throughout the porous matrix of the base feed.

The method for incorporating the pest control agent into the fish feed can include incorporating the pest control agent, such that the final concentration of pest control agent in the fish feed includes from about 0.01 gram pest control agent per kilogram of fish feed (g/kg) to about 1000 g/kg, or from about 0.01 g/kg, 0.1 g/kg, 1 g/kg, 2 g/kg, 3 g/kg, 4 g/kg, 5 g/kg, 6 g/kg, 7 g/kg, 8 g/kg, 9 g/kg or 10 g/kg, 20 g/kg, 30 g/kg, 40 g/kg, 50 g/kg, 60 g/kg, 70 g/kg, 80 g/kg, 90 g/kg, 100 g/kg, 250 g/kg, 500 g/kg, 750 g/kg, or 1000 g/kg, or any amount within a range of any of the forgoing. Concentrations are described herein in more detail in reference to the fish feeds.

The method for incorporating the pest control agent into the fish feed can include determining the final concentration of pest control agent that is incorporated as a part of the fish feed. The determination of the final concentration can include sampling the fish feed using various quantitative analytical methods. By way of example, the fish feed samples can be extracted by a process of overnight protein precipitation in methanol. Following extraction the sample can be cleaned with Supelm QuE Z-Sep+ (Sigma Aldrich, St. Louis, Missouri, USA) sorbent, which is a silica gel-based material having active zirconia-based phase, a particle size of approximately 50 μm, and a 70-angstrom (Å) pore size. The resulting extraction solution can be filtered through polytetrafluoroethylene filters having a pore size from 0.2 μm or greater. Analysis of the final concentration of pest control agent in the fish feed samples can be performed using high performance liquid chromatography with ultraviolet detection (IPLC-UV).

The methods herein can include extracting a neem extract rich in azadirachtin A, including those having from at least from 15% w/w to 33% w/w azadirachtin A, from at least 28% w/w to 31% w/w azadirachtin A, and from at least 29% w/w to 30% w/w azadirachtin A.

The uniformity in the amount of pest control agent within the fish feeds herein can be measured by the analytical methods herein and by assessing the coefficient of variation. Without wishing to be bound by any particular theory, it is understood that the coefficient of variation (CV) is the ratio of the standard deviation to the mean and shows the extent of variability in relation to the mean of the population. The coefficient of variation is represented as a percent (%) and can be used as a standardized measure of the variability within a population or data set. A higher CV indicates greater variability amongst the data and a lower CV indicates a lower variability amongst the data. It will be appreciated that in the context of mixing ingredients for food products, a CV of less than 10% indicates excellent mixing and no need for further optimization is required for the process. See, for example, Feed Manufacturing, Testing Mixer Performance, Bulletin M1172, Kansas State University Agriculture Experiment Station and Cooperative Extension Service. September 2017).

In a particular example herein, various batches of fish feed were prepared by providing a protein and starch mixture and subjecting it to and extrusion process to produce a base feed having a porous matrix distributed throughout. A pest control agent composition was created by mixing together an azadirachtin A-rich composition and one or more oils to create a suspension of azadirachtin A-rich composition and oil. The azadirachtin A-rich composition and oil suspension was applied to the base feed using a vacuum infusion process to distribute the azadirachtin A-rich composition and oil suspension throughout the porous matrix of the base feed. The coefficient of variation for the fish feeds was measured from the time of initial processing up to eight weeks post processing. Using the methods herein and a pest control rich in azadirachtin A, CV values of less than 10% (i.e., <10%) were consistently reported. The coefficient of variation for the batches of feed assayed are represented in Table 2. The average CV for all batches over the eight-week period is 2.35%.

TABLE 2
Coefficient of Variation of Fish Feed
as a Function of Storage Time

| Storage | | Mean Aza | Standard | Coefficient of |
|---|---|---|---|---|
| Time (weeks) | n | A (mg/g) | Deviation | Variation (%) |
| 0 | 6 | 2.01 | 0.09 | 4.38 |
| 1 | 3 | 1.90 | 0.02 | 1.26 |
| 2 | 3 | 1.88 | 0.05 | 2.61 |
| 3 | 3 | 1.87 | 0.03 | 1.34 |
| 4 | 3 | 1.92 | 0.07 | 3.42 |
| 8 | 3 | 1.97 | 0.02 | 1.12 |

### Fish Feeds Containing Azadirachtin A

The fish feeds herein can include those that are supplemented with the pest control agent azadirachtin A. The fish feeds can be administered to various fish as part of a fish feed diet to control arthropod pests within an aquaculture environment. The fish feeds can be at least partially coated on an exterior surface with an azadirachtin A-rich composition or completely coated on an exterior surface with an azadirachtin A-rich composition. In some aspects, the fish feeds herein can include an azadirachtin A-rich composition that is at least partially dispersed throughout the fish feed. In various aspects, a solid feed such as a base feed pellet, can further include an azadirachtin A-rich composition disposed on the surface or distributed throughout the fish feed, such as within an oil disposed within a porous matrix of on an exterior surface of the base feed pellet. In various aspects, the fish feeds herein can be at least partially coated on an exterior surface with an azadirachtin A-rich composition and further can have an azadirachtin A-rich composition at least partially dispersed throughout the fish feed. In various aspects, the fish feed can include one or more layers of azadirachtin A-rich composition on an exterior surface.

The fish feeds herein an azadirachtin A-rich composition at a concentration from about 0.01-100 grams per kilogram (g/kg) fish feed, about 90 g/kg fish feed, about 80 g/kg fish feed, about 70 g/kg fish feed, about 60 g/kg fish feed, about 50 g/kg fish feed, about 40 g/kg fish feed, about 30 g/kg fish feed, about 20 g/kg fish feed, about 0.01-10 g/kg fish feed, about 1-10 g/kg fish feed, about 2-9 g/kg fish feed, about 3-7 g/kg fish feed, about 4-6 g/kg fish feed, or about 5 g/kg fish feed.

In various aspects, the fish feeds herein can include an azadirachtin A-rich composition at a concentration from about 0.01 g azadirachtin A-rich composition per kilogram fish feed (g/kg), 0.05 g/kg fish feed, 0.1 g/kg fish feed, 0.2 g/kg fish feed, 0.3 g/kg fish feed, 0.4 g/kg fish feed, 0.5 g/kg fish feed, 0.6 g/kg fish feed, 0.7 g/kg fish feed, 0.8 g/kg fish feed, 0.9 g/kg fish feed, 1.0 g/kg fish feed. 1.25 g/kg fish feed, 1.5 g/kg fish feed, 1.75 g/kg fish feed, 2.0 g/kg fish feed, 2.25 g/kg fish feed, 2.5 g/kg fish feed, 2.75, g/kg fish feed, 3.0 g/kg fish feed, 5.0 g/kg fish feed. 5.25 g/kg fish feed, 5.5 g/kg fish feed, 5.75 g/kg fish feed, 6.0 g/kg fish feed, 6.25 g/kg fish feed, 6.5 g/kg fish feed, 6.75, g/kg fish feed, 7.0 g/kg fish feed, 7.0 g/kg fish feed. 7.25 g/kg fish feed, 7.5 g/kg fish feed, 7.75 g/kg fish feed, 8.0 g/kg fish feed, 8.25 g/kg fish feed, 8.5 g/kg fish feed, 8.75, g/kg fish feed, 9.0 g/kg fish feed, 9.25 g/kg fish feed, 9.5 g/kg fish feed, 9.75, g/kg fish feed, 10.0 g/kg fish feed, 15 g/kg fish feed, 20 g/kg fish feed, 25 g/kg fish feed, 30 g/kg fish feed, 35 g/kg fish feed, 40 g/kg fish feed, 45 g/kg fish feed, 50 g/kg fish feed, 55 g/kg fish feed, 60 g/kg fish feed, 65 g/kg fish feed, 70 g/kg fish feed, 75 g/kg fish feed, 80 g/kg fish feed, 85 g/kg fish feed, 90 g/kg fish feed, 95 g/kg fish feed, 100 g/kg fish feed, or any amount within a range of any of the forgoing concentrations.

It will be appreciated that the aforementioned azadirachtin A-rich composition concentrations equate to about from 0.001-10 (weight percent) % w/w azadirachtin A-rich composition to fish feed. In various aspects, the fish feeds herein can include an azadirachtin A-rich composition at from 0.001% w/w, 0.002% w/w, 0.003% w/w, 0.004% w/w, 0.005% w/w, 0.006% w/w, 0.007% w/w, 0.008% w/w, 0.009% w/w, 0.010% w/w, 0.020% w/w, 0.030% w/w, 0.040% w/w, 0.050% w/w, 0.060% w/w, 0.070% w/w, 0.080% w/w, 0.090% w/w, 0.10% w/w, 0.11% w/w, 0.12% w/w, 0.13% w/w, 0.14% w/w, 0.15% w/w, 0.16% w/w, 0.17% w/w, 0.18% w/w, 0.19% w/w, 0.20% w/w, 0.21% w/w, 0.22% w/w, 0.23% w/w, 0.24% w/w, 0.25% w/w, 0.26% w/w, 0.27% w/w, 0.28% w/w, 0.29% w/w, 0.30% w/w, 0.31% w/w, 0.32% w/w, 0.33% w/w, 0.34% w/w, 0.35% w/w, 0.36% w/w, 0.37% w/w, 0.38% w/w, 0.39% w/w, 0.40% w/w, 0.41% w/w, 0.42% w/w, 0.43% w/w, 0.44% w/w, 0.45% w/w, 0.46% w/w, 0.47% w/w, 0.48% w/w, 0.49% w/w, 0.50% w/w, 0.51% w/w, 0.52% w/w, 0.53% w/w, 0.54% w/w, 0.55% w/w, 0.56% w/w, 0.57% w/w, 0.58% w/w, 0.59% w/w, 0.60% w/w, 0.61% w/w, 0.62% w/w, 0.63% w/w, 0.64% w/w, 0.65% w/w, 0.66% w/w, 0.67% w/w, 0.68% w/w, 0.69% w/w, 0.70% w/w, 0.71% w/w, 0.72% w/w, 0.73% w/w, 0.74% w/w, 0.75% w/w, 0.76% w/w, 0.77% w/w, 0.78% w/w, 0.79% w/w, 0.80% w/w, 0.81% w/w, 0.82% w/w, 0.83% w/w, 0.84% w/w, 0.85% w/w, 0.86% w/w, 0.87% w/w, 0.88% w/w, 0.89% w/w, 0.90% w/w, 0.91% w/w, 0.92% w/w, 0.93% w/w, 0.94% w/w, 0.95% w/w, 0.96% w/w, 0.97% w/w, 0.98% w/w, 0.99% w/w, 1.0% w/w, 2.0% w/w, 3.0% w/w, 4.0% w/w, 5.0% w/w, 6.0% w/w, 7.0% w/w, 8.0% w/w, 9.0% w/w, or 10.0% w/w (i.e., weight azadirachtin A-rich composition to weight fish feed), or any amount within a range of any of the forgoing values.

In various aspects herein, the fish feeds can be administered to the fish having an azadirachtin A-rich composition at a concentration selected from the group including 0.05% w/w, 0.06% w/w, 0.07% w/w, 0.08% w/w, 0.09% w/w, 0.10% w/w, 0.11% w/w, 0.12% w/w, 0.13% w/w, 0.14% w/w, 0.15% w/w, 0.16% w/w, 0.17% w/w, 0.18% w/w, 0.19% w/w, 0.20% w/w, 0.30% w/w, 0.40% w/w, 0.50% w/w, 0.60% w/w, 0.70% w/w, 0.80% w/w, 0.90% w/w, or 1.0% w/w (i.e., weight azadirachtin A-rich composition to weight fish feed), or a range within any of the forgoing concentrations.

Azadirachtins can be relatively unstable in water, however when they are a component of a fish feed, such as dispersed throughout or coated thereon, the azadirachtins, including azadirachtin A, are rendered at least temporarily stable such that they can exhibit their full biological activity during feeding. Any fish feed that falls to the ocean floor will degrade upon prolonged exposure to water. In addition, it should be noted that azadirachtin A, or any of the neem extract agents of the present disclosure exhibit minimal risk of toxic effects on fish or humans and are therefore safe to use in both wild fish and farmed fish stocks. While arthropods and other invertebrates are sensitive to the active ingredient (i.e. azadirachtin A), higher organisms, including mammals, are unaffected. Furthermore, since azadirachtin A or any neem extract as described herein are readily soluble in water, they do not reside and accumulate in fish. Rather, once administration has ceased, the pest control agent can quickly lose effectiveness, as it is metabolized, degraded, and/or excreted. In the case of farmed fish stocks, this ensures a little to no withdrawal period to harvest following administration of a fish feed or composition as described herein.

The fish feed provided herein can include an azadirachtin A-rich composition together with one or more other agents. The one or more or other agents can include anti-ectoparasitic agents, antimicrobial agents (e.g., antibiotic, antibacterial, antifungal, antiviral agents), antiparasitic agents (e.g., anti-endoparasitic agents or anti-ectoparasitic agents), or antiprotozoal agents. The one or more other agents can be mixed with or coated on, or layered within, the fish feed. The one or more other agents can be provided separately (e.g., either in liquid or solid form) and can be administered separately (e.g., before or after) or concurrently with (e.g., during) a fish feed.

### Pest Control Agent Compositions

The present disclosure further provides a pest control agent composition for administration to fish, where the pest control agent composition can include one or more pest control agents. It will be appreciated that the pest control agent compositions are not a fish feed and are intended for separate or supplemental administration to fish in addition to a fish feed. The pest control agent compositions can be provided separately for administration before, during, or after administration of the fish feeds. Accordingly, in various aspects, the pest control agent compositions herein can be suitable for use in some aspects as a form of veterinary medicinal product or dietary supplement for reducing, preventing, or controlling pest infections or pest infestations in fish. The pest control agent compositions can be administered to the fish at the concentrations described elsewhere herein. For example, the pest control agent compositions herein can be administered at from about 0.01 grams pest control agent per kilogram of fish feed (g/kg) to about 100 g/kg, as described elsewhere herein. In various aspects, the pest control agent composition includes a neem extract rich in azadirachtin A.

The pest control agent compositions herein can include azadirachtin extracts rich in azadirachtin A. The pest control agent composition can include a liquid, solid, or semi-solid form, and further can include one or more of an excipient, diluent, carrier, vitamins, minerals, or combinations thereof. The pest control agent compositions can be in the form of a dietary supplement that is provided as any of granules, flakes, pellets, powders, tablets, pills, capsules, and the like. In various aspects, the pest control agent compositions herein can be formed into many shapes and sizes. In various aspects, the fish feeds herein can be in the shape of a triangle, a square, a rectangle, a sphere, a diamond, a cylinder, a pellet, a clover, an amorphous shape, and the like. The fish feeds can be formed by a process including one or more of extrusion, retort, cold-pressing, high-pressure processing, and the like.

Alternatively, the pest control agent composition can be provided in a form that is edible by fish but that does not provide nutrition to the fish. By way of example, the pest control agent composition can include a veterinary medicinal product that can include substances or combinations of substances to manage or prevent diseases in fish. The pest control agent composition can also be formulated for parenteral administration. Thus, the pest control agent composition can include pharmaceutically acceptable carriers, diluents, or excipients, or combinations thereof. Furthermore, the pest control agent composition can be sterile.

The pest control agent compositions herein can be included in one or more types of fish feed designed for mixing with another composition, such as a base feed. The pest control agent composition can be in the form of a premix, a concentrate, a base mix, a supplement, a top dress, liquid drench, or a combination thereof.

The pest control agent in the pest control agent compositions herein can include one or more agents for reducing, preventing, or controlling an infection or infestation caused or contributed by one or more endoparasite or ectoparasite pests, including any type of worms, helminths, flukes, lice, mites, bacteria, viruses, fungi, and protozoa, as described elsewhere. Each pest control agent included in the pest control agent compositions can be individually capable of reducing, preventing, or controlling one or more of a parasitic, bacterial, viral, fungal, or protozoal infections or infestations. By way of example, the pest control agent compositions herein can include those exhibiting one or more inhibitory effects, including an antiparasitic effect, an antibacterial effect, an antiviral effect, an antifungal effect, an antiprotozoal effect, or any combinations thereof.

A pest control agent composition can be administered before during or after the administration of any of the fish feeds. In some aspects, the pest control agent compositions can be administered with fish feed that does not contain a pest control agent. In some aspects, the pest control agent compositions can be administered in conjunction with fish feed that does contain a pest control agent. When used in conjunction with fish feed that does contain a pest control agent, the separate pest control agent composition can include the same pest control agent as in the fish feed or it can be a different pest control agent than in the fish feed. When used in conjunction with fish feed that does contain a pest control agent, the separate pest control agent composition can be the same concentration as the pest control agent in the fish feed or it can be a different concentration than the pest control agent in the fish feed. The pest control agent compositions herein can be included in the diet of fish in the form of a veterinary medicinal product or dietary supplement to any complete and balanced fish feed or can be provided as a component of a complete fish feed.

### Target Fish and Pests

The fish feeds provided herein can be fed as a fish feed diet or used to feed any fish that is susceptible to infection or infestation by one or more pest. For example, the fish feed can be used in aquaculture as a component of a diet fed to any farmed fish including, for example, commercially relevant fish species. For example, the fish feeds provided herein can form part of diet fed to any of freshwater fish, brackish fish, or saltwater fish. The fish feeds can be used as a component of a diet fed to any species belonging to the families Cyprinidae, Cichlidae, Pangasiidae, Sciaenidae, Serranidae, Carangidae, Sparidae, Lateolabracidae, Moronidae, Mugilidae, Cypriniformes, Latidae, Eleotridae, Tilapiini and Salmonidae. As such, the fish feeds herein can be used to feed species belonging to any of the genera within these families and in particular, those species that are farmed for human or animal consumption. For example, and without limitation, the fish feeds described herein can be used to feed species belonging to the genera Salmo and/or Oncorhynchus. In particular, the fish feeds herein can be used to control pests in populations of wild or farmed salmon or trout species, including, for example, any of Atlantic salmon (Salmo salar), Pacific salmon, Char, or Rainbow trout. Moreover, the fish feed can be used as a pest control agent for other fish species within the aquaculture industry such as sea bass, bream, grouper, pompano, and tuna, as well as in the pet and decorative fish industries, for example for pest control in goldfish (Carassius auratus).

Pests of the target fish herein can include ectoparasites including species belonging to the phylum Arthropoda. Susceptible arthropods include various copepods that include many species of sea lice that inhabit fish hosts. By way of example, in the case of fish belonging to the family Salmonidae (e.g., Salmo and/or Oncorhynchus spp.), the pest control agent of the fish feed provided herein can control sea lice infections, sea lice infestations, copepod infections, copepod infestations, or any combinations thereof. The pest control agent present in the fish feeds provided herein can control ectoparasites including parasitic crustaceans, also referred to as copepods, belonging to Argulus ssp. or Caligus ssp. In particular, the pest control agent for use in the fish feed described herein can be effective at controlling copepod infections and infestations of one or more types of farmed fish. As used herein, the term “copepod” refers to a group of crustaceans found in fresh water and in seawater, and which have one or more parasitic phases of their life cycle. Unless otherwise noted, the term copepod can refer to any of the various species of sea lice as described herein.

A species of copepod that is an ectoparasite of Atlantic salmon belongs to the Lepeophtheirus genus and is known as the salmon louse, Lepeophtheirus salmonis. As used herein, it will be appreciated that the term “sea louse” refers to the singular form and the term “sea lice” refers to the plural form. Each term can be used interchangeably unless otherwise noted. Lepeophtheirus salmonis are a species of copepod ectoparasites that primarily live on salmon, including Atlantic and Pacific salmon, and sometimes on sea trout. Other types of copepod ectoparasites infecting fish belonging to the family Salmonidae include Caligus clemensi, Caligus elongatus, and Caligus rogercresseyi. Sea lice, including those from the genera Lepeophtheirus and Caligus, are ectoparasites which feed off the blood, mucus, muscle, and skin of various salmon species.

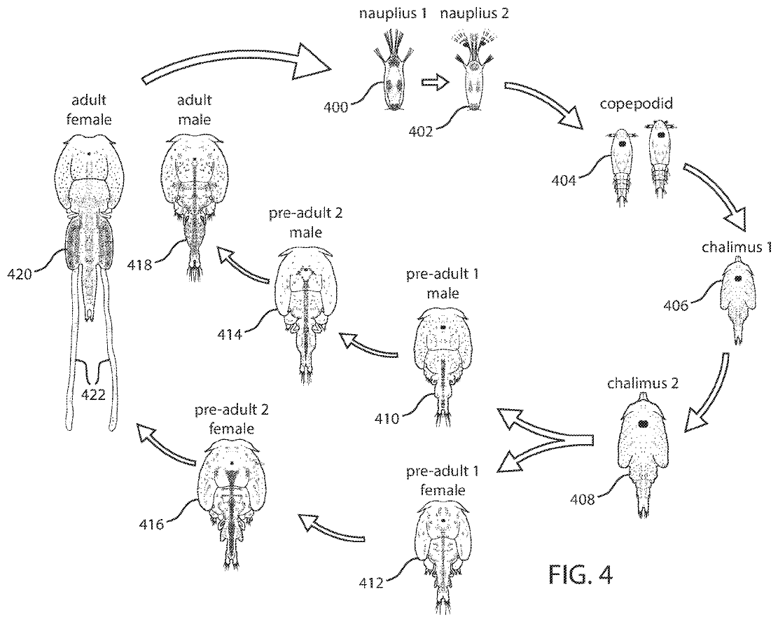

Referring now to FIG. 4, the life cycle of Lepeophtheirus salmonis (i.e., L. salmonis) is shown in accordance with various aspects herein. Various aspects of FIG. 1 have been adapted from Sea Lice Research Centre, 2020, “SLRC—Life cycle of the salmon louse (Lepeophtheirus salmonis)”, https://doi.org/10.18710/GQTYYL, DataverseNO, V1. The life cycle of L. salmonis is complex and consists of eight distinct life stages. The nauplius stage begins upon egg hatching and includes two distinct stages, including the nauplius 1 stage and the nauplius 2 stage, shown as 400 and 402, respectively. The nauplius 1 stage 400 and nauplius 2 stage 402 of L. salmonis drift passively in water where, in general, both stages are about 0.5 mm to 0.7 mm in length and they are relatively translucent in color. The nauplius 1 stage 400 and nauplius 2 stage 402 exist for about 52 hours and 175 hours, respectively, in temperatures of up to about 15 °C. Upon molting from the nauplius 2 stage 402, L. salmonis transitions into the infective copepodid stage 404 at approximately 10 days of age, with the understanding that development at any life stage can depend on a number of environmental factors, including but not to be limited to temperature, salinity, light hours, and the like. L. salmonis in the copepodid stage 404 are approximately from 0.7 mm to 1.0 mm in length. In the copepodid stage 404, L. salmonis generally attaches to fish along its fins or scales. After attachment, L. salmonis further molts to the chalimus 1 stage 406. In the chalimus 1 stage 406, L. salmonis attaches to the host fish more firmly by way of a frontal filament. The chalimus 1 life stage typically lasts approximately 10 days. The L. salmonis life cycle continues with a molting from chalimus 1 stage 406 to the chalimus 2 stage 408 while remaining firmly attached to the fish. During the chalimus 1 stage 406 and the chalimus 2 stage 408, the L. salmonis is an average length from 1.0 mm to 2.5 mm. The chalimus 2 life stage typically lasts approximately 10 days.

Following both chalimus stages, L. salmonis molts further into the pre-adult stage where L. salmonis becomes mobile and able to swim or move around the fish surface. At the pre-adult stage, sexual development begins to differentiate between the females and males. The pre-adult stage consists of 2 distinct stages, where males develop from chalimus 2 stage 408 into the pre-adult 1 male stage 410 and females develop into the pre-adult 1 female stage 412. The females spend approximately 10 days as the pre-adult 1 female stage 412 and spend approximately 12 days at the pre-adult 2 female stage 416. The males spend approximately 8 days as the pre-adult 1 male stage 410 and spend approximately 9 days at the pre-adult 2 male stage 414. From the pre-adult 1 stages, the sexes continue through development into the pre-adult 2 male stage 414 and the pre-adult 2 female stage 416. During the pre-adult stages, L. salmonis grows to an average length of about 2.5 mm to 3.5 mm. In the final adult stage, the adult male 418 and adult female 420 are distinguishable by size and phenotypic characteristics. Namely, the adult female 420 is from 8 mm to 20 mm in length, including two egg strings 422 visible off the posterior aspect of the organism. In contrast, the adult male 418 is from 5 mm to 7 mm in length.

On average, L. salmonis can live for approximately up to 215 days for a full life cycle. It will be appreciated that each stage of the L. salmonis life cycle can be dependent on temperature, salinity of the water, water currents, pollution levels, and various additional environmental factors. Thus, the complete life cycle of L. salmonis can be from 32 days up to 215 days depending on the fluctuations in such external factors.

Thus, in various aspects, the disclosure herein provides a fish feed including a pest control agent capable of any of reducing, preventing, or controlling Lepeophtheirus or Caligus infections or infestations. A Lepeophtheirus infection or infestation can be caused or contributed by the salmon louse, Lepeophtheirus salmonis. A Caligus infection or infestation can be caused or contributed to by the sea lice, Caligus clemensi, Caligus elongatus, and Caligus rogercresseyi. In various aspects, the fish feed can be for species belonging to the family Salmonidae. For example, the fish feeds herein can be a Salmo and/or Oncorhynchus spp. fish feed. However, it will be appreciated that the fish feeds herein can be given to any fish that is susceptible to a Lepeophtheirus or Caligus infection or infestation.

### Methods of Administration of Pest Control Agents to Fish

The pest control agents herein can be administered to fish in the fish feeds and pest control agent compositions. Management methods that utilize the pest control agents within fish feeds can be referred to as in-feed agent delivery methods. Thus, the present disclosure provides in-feed agent delivery methods for reducing, preventing, or controlling pests. It will be appreciated that an in-feed agent delivery method is not a process that applies the pest control agents topically to the target pests. In-feed agent delivery methods herein can further utilize pest control agents in a non-feed form such as a veterinary medicinal product or dietary supplement. Thus, the present disclosure further provides veterinary medicinal products or dietary supplements as agents for reducing, preventing, or controlling pests.

The pest control agents that are not included in fish feed can be administered to the fish in a separate pest control agent composition as a complement to fish feed, such as in the form of a veterinary medicinal product or a dietary supplement. The fish feed can be administered at the same time or separately from a pest control agent composition. It should be noted that the various pest control agents herein can be administered to fish that are sick, fish that are infested with parasites, fish that are otherwise healthy in order to prevent parasitic infection, or fish that are less aggressive due to a different infection or condition not associated with a pest infection or infestation. It will be understood that fish that are sick or less aggressive may eat less and therefore may consume lower concentrations of the pest control agent. Thus, management methods that are based on the use of both fish feeds and pest control agent compositions can be particularly useful for managing fish whose appetites are affected by illness, infection, infestation, or being a less aggressive fish that generally eats less fish feed. Moreover, in less aggressive, or low feeding fish, the concurrent use of a pest control agent composition with fish feed supplemented with a pest control agent can boost or ensure the correct pest control agent concentration is administered to fish.

The concentration of pest control agent added to fish feeds or pest control agent compositions herein can be an amount effective to achieve the desired modulation of the behavior, development, or mortality of the pests as discussed elsewhere herein. It will be appreciated that the exact amount of pest control agent to be added to a fish feed or pest control agent compositions herein can vary depending on, for example, the species of fish, the number of fish to be fed, the extent of the infection or infestation, and the like. Other factors that influence the amount of pest control agent added to the fish feeds or pest control agent compositions include, for example, the presence of possible competitors for the feed (i.e. other non-target animal species that can eat the fish feed), the type of pest to be controlled, the age and maturity of the pests, the age and maturity of the fish, the season, the water type (e.g., pH, salinity, purity, temperature), and the aggressiveness of the fish. It will be appreciated that the concentration of pest control agents added to a fish feed or pest control agent composition herein can include an amount effective to achieve a desired effect to modulate the behavior and development of the pests, where the amount effective includes one or more concentrations or ranges of concentrations as recited herein. It will be appreciated that the effective amount effective can be determined by performing a comparison to a control fish or group of fish not fed the pest control agents.

The fish feeds and pest control agent compositions can be formulated such that the concentration of the pest control agent administered to the fish through the fish feed or pest control agent compositions can be approximately 0.01-100 mg pest control agent per kg body weight/day (mg/kg/day), 1-90 mg/kg/day, 1-80 mg/kg/day, 1-70 mg/kg/day, 1-60 mg/kg/day, 5-50 mg/kg/day, 10-40 mg/kg/day, 15-35 mg/kg/day, 20-30 mg/kg/day, 0.01-10 mg/kg/day, or 0.01-5.0 mg/kg/day. In various aspects, the pest control agent is administered to the fish in the fish feed at a targeted concentration from 0.01 mg to 5.0 mg azadirachtin A per kg body weight per day. In various aspects, the pest control agent is administered to the fish in the fish feed at a targeted concentration from 1.5 mg to 2.5 mg azadirachtin A per kg body weight per day. In some aspects, the pest control agent is administered to the fish in the fish feed at a targeted concentration from 2.6 mg to 5.0 mg azadirachtin A per kg body weight per day.

It will be appreciated that the amount of pest control agent administered to the fish can include an amount effective to produce an inhibitory effect against one or more pests within a range of approximately 0.01-100 mg/kg/day (e.g., mg pest control agent/kg body weight/day) includes at least 0.01 mg/kg/day, 0.02 mg/kg/day, 0.03 mg/kg/day, 0.04 mg/kg/day, 0.05 mg/kg/day, 0.06 mg/kg/day, 0.07 mg/kg/day, 0.08 mg/kg/day, 0.09 mg/kg/day, 0.10 mg/kg/day, 0.20 mg/kg/day, 0.30 mg/kg/day, 0.40 mg/kg/day, 0.50 mg/kg/day, 0.60 mg/kg/day, 0.70 mg/kg/day, 0.80 mg/kg/day, 0.90 mg/kg/day, 1.0 mg/kg/day, 2.0 mg/kg/day, 3.0 mg/kg/day, 4.0 mg/kg/day, 5.0 mg/kg/day, 6.0 mg/kg/day, 7.0 mg/kg/day, 8.0 mg/kg/day, 9.0 mg/kg/day, 10.0 mg/kg/day, 11.0 mg/kg/day, 12.0 mg/kg/day, 13.0 mg/kg/day, 14.0 mg/kg/day, 15.0 mg/kg/day, 16.0 mg/kg/day, 17.0 mg/kg/day, 18.0 mg/kg/day, 19.0 mg/kg/day, 20.0 mg/kg/day, 25.0 mg/kg/day, 30.0 mg/kg/day, 35.0 mg/kg/day, 40.0 mg/kg/day, 45.0 mg/kg/day, 50.0 mg/kg/day, 55.0 mg/kg/day, 60.0 mg/kg/day, 65.0 mg/kg/day, 70.0 mg/kg/day, 75.0 mg/kg/day, 80.0 mg/kg/day, 85.0 mg/kg/day, 90.0 mg/kg/day, 95.0 mg/kg/day, or 100.0 mg/kg/day, or any amount within a range of any of the forgoing values. Any of the aforementioned amounts effective to produce an inhibitory effect can be utilized in the targeted concentrations described herein.

A fish feed or pest control agent composition can be administered for a period of time for as long as required to achieve the desired inhibitory effect. For example, the pest control agent composition or fish feed can be administered over about a 10 to 20 days, or about 14-18 days. It will be appreciated that, the pest control agent composition, the fish feed, or both, can be administered for consecutive days for 10 days, 11 days, 12 days, 13 days, 14 days, 15 days, 16 days, 17 days, 18 days, 19 days, or 20 days, or for any number of days falling within a range of any of the forgoing. In various aspects, the pest control agent composition or fish feeds herein can be administered for a longer period of time, such as past 20 days. In various aspects, the fish feed or pest control agent can be administered for at least 11 days. In various aspects, the fish feed or pest control agent can be administered for at least 14 days. It should be understood that the time required for administration of the pest control agent composition or fish feeds herein can be of a variable length in order to target the developmental life stages of the pests present in a fish population, for water temperature, pest control agent concentration, or any combinations thereof. In some aspects, the pest control agents herein could be administered prophylactically in the diet of fish at an amount effective to prevent a pest infection or infestation from taking hold within a population of fish. It will further be appreciated that the pest control agents herein could be administered prophylactically in the diet for any period of time during the fish life cycle, such as from stocking to harvest, seasonally, or during an infection or infestation outbreak within a population or within a nearby farm infection or infestation outbreak.

In various management methods, the pest control agents herein can be administered for non-consecutive days, where the pest control agent is administered for a predetermined period of time followed by a rest period, and then administered again for a predetermined period of time and followed by a rest period, and so on. By way of example, in some aspects, the pest control agent can be administered for three out of every 10 days. In other aspects, the pest control agent can be administered for seven out of every 14 days. The method for administering the pest control agent for predetermined period of time followed by a rest period can be repeated for as long as desired or until a pest infection or infestation is reduced, prevented, or controlled. It will be appreciated that the pest control agent can be administered for a predetermined period of time, including from 1 day, 2 days, 3 days, 4 days, 5 days, 6 days, 7 days, 8 days, 9 days, or 10 days out of every 5 days to 30 days of rest in between administration.

During the period of administration, the pest control agent composition or fish feed can be administered as many times per day as required to achieve the inhibitory effect. For example, the pest control agent composition or fish feeds described herein can be administered about 1, 2, 3, 4, 5, 6, 7, 8, 9, 10, or more times a day.

It should be understood that the definitions described herein apply to all aspects as described unless otherwise stated.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference is to be considered supplementary to that of this document; for irreconcilable inconsistencies, the usage in this document controls.

Values expressed in a range format are to be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” is to be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis (% w/w or w/w %) is also referred to as weight percent (wt. %) or percent by weight (% wt.) herein.

Exemplary embodiments of the present invention are as follows:

Embodiment 1: A fish feed comprising:
a base feed comprising a porous matrix distributed throughout; and
a pest control agent composition, the pest control agent composition comprising a pest control agent and one or more oils;
wherein the pest control agent composition is distributed throughout the porous matrix of the base feed.

Embodiment 2: The fish feed of Embodiment 1, wherein the pest control agent comprises neem extract rich in azadirachtin A.

Embodiment 3: The fish feed of Embodiment 2, wherein the neem extract rich in azadirachtin A comprises at least from 15 wt. % to 33 wt. % azadirachtin A.

Embodiment 4: The fish feed of any of Embodiments 1-3, wherein the one or more oils comprise one or more of soy oil, grape seed oil, canola oil, sunflower seed oil, safflower oil, rapeseed oil, hydrogenated rapeseed oil, linseed oil, camelina oil, flaxseed oil, coconut oil, fish oil, poultry oil, or fish oil stearin, or fractions thereof.

Embodiment 5: The fish feed of any of Embodiments 1-4, wherein the porous matrix is formed by extrusion processing of raw materials comprising a protein and a starch.

Embodiment 6: The fish feed of any of Embodiments 1-5, wherein the pest control agent composition is applied to the base feed via a vacuum infusion process.

Embodiment 7: The fish feed of any of Embodiments 1-6, wherein the pest control agent is at least partially distributed throughout the porous matrix of the base feed.

Embodiment 8: The fish feed of any of Embodiments 1-7, wherein the pest control agent composition is further coated on an exterior surface of the base feed.

Embodiment 9: The fish feed of any of Embodiments 1-8, wherein the fish feed comprises between about 15 wt. % and about 65 wt. % protein and between about 10 wt. % and about 45 wt. % fat; and
wherein the fish feed pellet comprises land-animal protein, fish meal, plant-based protein, or combinations thereof.

Embodiment 10: The fish feed of any of Embodiments 1-9, wherein the base feed comprises one or more of pellets, extruded nuggets, steam pellets, flakes, tablets, or powders.

Embodiment 11: The fish feed of any of Embodiments 1-10, wherein the pest control agent comprises a solid form suspended in one or more oils.

Embodiment 12: A method for making a fish feed comprising:
providing a protein and starch mixture and subjecting it to and extrusion process to produce a base feed having a porous matrix distributed throughout;
creating a pest control agent composition by mixing together a pest control agent and one or more oils to create a suspension;
applying the pest control agent composition to the base feed using a vacuum infusion process to distribute the pest control agent composition throughout the porous matrix of the base feed.

Embodiment 13: The method of Embodiment 12, wherein the pest control agent comprises a neem extract rich in azadirachtin A comprising at least from 15 wt. % to 33 wt. % azadirachtin A.

Embodiment 14: The method of any of Embodiments 12 or 13, wherein the one or more oils comprise one or more of soy oil, grape seed oil, canola oil, sunflower seed oil, safflower oil, rapeseed oil, hydrogenated rapeseed oil, linseed oil, camelina oil, flaxseed oil, coconut oil, fish oil, poultry oil, or fish oil stearin, or fractions thereof.

Embodiment 15: The method of any of Embodiments 12-14, wherein the extrusion process comprises extrusion cooking of raw materials comprising a protein and a starch.

Embodiment 16: The method of any of Embodiments 12-15, wherein the method of producing a base feed by an extrusion process followed by applying a pest control agent composition by a vacuum infusion process produces a fish feed having a coefficient of variation of less than 10%.

Embodiment 17: A fish feed product according to the process of any of Embodiments 12-16.

Embodiment 18: A fish feed for reducing, preventing, or controlling a parasite infection or infestation in fish, wherein the fish feed comprises:
a base feed comprising a porous matrix distributed throughout; and
a pest control agent composition, the pest control agent composition comprising a pest control agent and one or more oils;
wherein the pest control agent composition is distributed throughout the porous matrix of the base feed.

Embodiment 19: The fish feed of Embodiment 18, wherein the pest control agent comprises neem extract rich in azadirachtin A.

Embodiment 20: The fish feed of Embodiment 19, wherein the neem extract rich in azadirachtin A comprises at least from 15 wt. % to 33 wt. % azadirachtin A.

1.-15. (canceled)

16. A fish feed comprising:
a base feed comprising a porous matrix distributed throughout; and
a pest control agent composition, the pest control agent composition comprising a pest control agent and one or more oils;
wherein the pest control agent composition is distributed throughout the porous matrix of the base feed.

17. The fish feed of claim 16, wherein the pest control agent comprises neem extract rich in azadirachtin A.

18. The fish feed of claim 17, wherein the neem extract rich in azadirachtin A comprises at least from 15 wt. % to 33 wt. % azadirachtin A.

19. The fish feed of claim 16, wherein the one or more oils comprise one or more of soy oil, grape seed oil, canola oil, sunflower seed oil, safflower oil, rapeseed oil, hydrogenated rapeseed oil, linseed oil, camelina oil, flaxseed oil, coconut oil, fish oil, poultry oil, or fish oil stearin, or fractions thereof.

20. The fish feed of claim 16, wherein the porous matrix is formed by extrusion processing of raw materials comprising a protein and a starch.

21. The fish feed of claim 16, wherein the pest control agent composition is applied to the base feed via a vacuum infusion process.

22. The fish feed of claim 16, wherein the pest control agent is at least partially distributed throughout the porous matrix of the base feed.

23. The fish feed of claim 16, wherein the pest control agent composition is further coated on an exterior surface of the base feed.

24. The fish feed of claim 16, wherein the fish feed comprises between about 15 wt. % and about 65 wt. % protein and between about 10 wt. % and about 45 wt. % fat; and
wherein the fish feed pellet comprises land-animal protein, fish meal, plant-based protein, or combinations thereof.

25. The fish feed of claim 16, wherein the base feed comprises one or more of pellets, extruded nuggets, steam pellets, flakes, tablets, or powders.

26. The fish feed of claim 16, wherein the pest control agent comprises a solid form suspended in one or more oils.

27. A method for making a fish feed comprising:
providing a protein and starch mixture and subjecting it to and extrusion process to produce a base feed having a porous matrix distributed throughout;
creating a pest control agent composition by mixing together a pest control agent and one or more oils to create a suspension;
applying the pest control agent composition to the base feed using a vacuum infusion process to distribute the pest control agent composition throughout the porous matrix of the base feed.

28. The method of claim 27, wherein the pest control agent comprises a neem extract rich in azadirachtin A comprising at least from 15 wt. % to 33 wt. % azadirachtin A.

29. The method of claim 27, wherein the one or more oils comprise one or more of soy oil, grape seed oil, canola oil, sunflower seed oil, safflower oil, rapeseed oil, hydrogenated rapeseed oil, linseed oil, camelina oil, flaxseed oil, coconut oil, fish oil, poultry oil, or fish oil stearin, or fractions thereof.

30. The method of claim 27, wherein the extrusion process comprises extrusion cooking of raw materials comprising a protein and a starch.

31. The method of claim 27, wherein the method of producing a base feed by an extrusion process followed by applying a pest control agent composition by a vacuum infusion process produces a fish feed having a coefficient of variation of less than 10%.

32. A fish feed product according to the process of any of claims 16-31.

33. A fish feed for reducing, preventing, or controlling a parasite infection or infestation in fish, wherein the fish feed comprises:
a base feed comprising a porous matrix distributed throughout; and
a pest control agent composition, the pest control agent composition comprising a pest control agent and one or more oils;
wherein the pest control agent composition is distributed throughout the porous matrix of the base feed.

34. The fish feed of claim 33, wherein the pest control agent comprises neem extract rich in azadirachtin A.

35. The fish feed of claim 33, wherein the neem extract rich in azadirachtin A comprises at least from 15 wt. % to 33 wt. % azadirachtin A.
